(19) United States
(12) Patent Application Publication
(10) Pub. No.: US 2006/0148099 A1
(43) Pub. Date: Jul. 6, 2006

(54) Sensor device for determining protein aggregation

(75) Inventors: Neville John Freeman, Tarporley (GB); Gerard Anthony Ronan, Worcester (GB); Marcus Swann, Lymm (GB); David Allsop, Lancashire (GB); Susan Moore, Lancashire (GB); Jon Popplewell, Manchester (GB)

Correspondence Address: WELLS ST. JOHN P.S., 601 W. FIRST AVENUE, SUITE 1300, SPOKANE, WA, 99201, US

(73) Assignee: Farfield Sensors Limited, Manchester Airport (GB)

(21) Appl. No.: 10/533,018

(22) PCT Filed: Oct. 31, 2003
(86) PCT No.: PCT/GB03/04731

§ 371 Date: Nov. 5, 2005

(30) Foreign Application Priority Data
Nov. 1, 2002 (GB) 0225491.0

(51) Int. Cl. G06F 19/00; C12M 1/34; G01N 33/543; G01N 33/48

(52) U.S. Cl. 436/518; 435/287.2; 702/19

(57) ABSTRACT

The present invention relates to a sensor device and method for determining the extent of aggregation of a protein such as beta amyloid in a fluid (e.g. a bodily fluid).

The present invention relates to a sensor device and method for determining the extent of aggregation of a protein such as beta amyloid in a fluid (e.g a bodily fluid).

Many diseases (particularly diseases of the brain) are the result of undesirable aggregation of certain peptides and proteins. Examples of such diseases include Alzheimer's disease (AD), Parkinson's disease and Huntingdon's disease. Other diseases in which such mechanisms are important include diabetes.

In the case of AD, the protein APP protrudes through the membranes of neurones (partly inside and partly outside the cell) and whilst being embedded in the membrane, enzymes cleave it in two so as to create a beta amyloid fragment. In a normal subject, it is thought that certain substances may bind to the APP fragments keeping them in solution but in a subject suffering from AD, the beta amyloid fragment is caused to drop out of solution and form insoluble plaques. In general, there is uncertainty as to how beta amyloid effects neurones but it has been observed that the protein is toxic to neurones. For example, it has been demonstrated that hippocampus neurones die when beta amyloid is added to a cell culture. Other studies have suggested that beta amyloid breaks into fragments releasing free radicals that attack neurones. Further research has postulated that beta amyloid may form tiny channels in neuron membranes allowing uncontrolled amounts of calcium into the cell or alternatively may disrupt potassium channels (which in turn could also effect calcium levels). Other studies have suggested that the age of the beta amyloid is important in determining toxicity to neurones so that simply detecting its presence is largely unhelpful as it may not be possible to differentiate beta amyloid in a state that is toxic from beta amyloid in a state that is non-toxic.

The present invention is based on the recognition that exploiting a sensor device sensitive to mass changing events at the molecular level to detect the extent of aggregation of a protein to which it is exposed may be a useful ex vivo (or even potentially in vivo) diagnostic tool where a critical extent of aggregation is clinically associated with the onset of (or a high risk of developing) a disease. More particularly, the present invention relates to a sensor device and method for detecting the extent of aggregation of a protein (such as beta amyloid) in a sample of bodily fluid using an antibody capable of binding a sequence of its amino acids.

Thus viewed from one aspect the present invention provides a sensor device for determining the extent of aggregation of a protein in a fluid having a sensor component capable of exhibiting a measurable response to a change in a localised environment caused by the introduction of the fluid, wherein a surface of said sensor component exposed to the fluid is provided with a specific binding partner to the protein rendering the measurable response to the change in the localised environment discriminatory to the extent of aggregation of the protein.

For the avoidance of doubt, the term “protein” used herein is intended to cover peptides. The specific binding partner is typically an antibody or aptamer.

In a preferred embodiment, the protein is beta amyloid. Particularly preferably the specific binding partner to beta amyloid is an optionally biotinlyated antigen (e.g commercially available biotinylated 6E-10).

The fluid may be a bodily fluid (e.g a human or non-human bodily fluid). The bodily fluid may be selected from the group consisting of blood serum, plasma and CSF. Preferably the bodily fluid is self-extracted. Preferably the bodily fluid is extracted remotely. By determining the extent of aggregation of a protein such as beta amyloid (e.g an early state of aggregation when only a few protein or peptide units have associated with each other), it may be possible (for example) to detect the onset of (or a high risk of developing) a disease in the subject from which the bodily fluid is derived. The disease may be any disease associated with aggregated proteins but particularly those associated with amyloidogenic processes.

Viewed from a further aspect the present invention provides a method for determining the extent of aggregation of a protein in a fluid, said method comprising:

(A) providing a sensor device as herein defined;

(B) irradiating the sensor component with electromagnetic radiation to generate an output;

(C) introducing the fluid into the localised environment;

(D) measuring the response of a characteristic of the output; and

(E) relating the response of the characteristic of the output to the extent of aggregation of the protein.

The sensor component may be capable of exhibiting a measurable response in a parameter selected from effective refractive index, a dielectric constant, aviscoelastic property, a frequency of oscillation, a thermal absorption/desorption parameter, the permeability, the absorption of energy or of energetic particles (such as x-rays, gamma rays, β-rays, electrons, neutrons, ions, light, microwaves, acoustic waves) or the particle size. For example, the sensor device may be one or more of the following types: surface plasmon resonance sensor devices, resonant mirror sensor devices, acoustic sensor devices (such as quartz crystal and surface acoustic wave devices (by using frequency decay techniques for example)) or electrical sensor devices (capable of measuring impedance at (for example) RF or microwave frequencies). Preferably the parameter is the effective refractive index.

The surface of the sensor component may be derivatised for the purposes of attaching or absorbing the specific binding partner. For example, the surface of the sensor component may comprise an absorbent material (e.g a polymeric material such as polymethylmethacrylate, polysiloxane, poly-4-vinylpyridine). For example, the surface of the sensor component may comprise a porous silicon material (e.g trimethoxyaminosilane) capable of being biofunctionalised with the specific binding partner.

Specific binding partner (e.g. antibody) preparation may be undertaken using standard techniques which are familiar to those skilled in the art enabling (for example) an antibody to be raised to monomeric and low oligomer materials of aggregated peptides. The desired specific binding partner may be attached to or absorbed in the sensor component in a conventional manner.

In a preferred embodiment, step (C) further comprises:

(C′) introducing an amount of the specific binding partner into the localised environment.

This embodiment constitutes a so-called “sandwich assay” and may advantageously improve the sensitivity of the method of the invention relative to a direct assay. The specific binding partner is typically present in solution and may be immobilised in accordance with techniques familiar to those skilled in the art.

Alternatively an additional proportion of fluid may be added to effectively measure the degree of secondary aggregation that occurs which is dependent upon the maturity of the protein aggregate captured initially in step (C).

Alternatively an additional proportion of an aggregate monomer may be added to effectively measure the degree of secondary aggregation that occurs which is dependent upon the maturity of the protein aggregate captured initially in step (C).

The derivatised surface of the sensor component may be biotinylated using standard reagents and optionally blocked with aconventional blocking solution (e.g. TWEEN®, gelatine and/or gamma globulin). This advantageously prevents binding of protein aggregates below a threshold size and excludes secondary species. The same effect may be achieved with surface loading or membrane modification or other such techniques.

Before step (C), step (B) of the method may comprise:

(B′) irradiating the sensor component with electromagnetic radiation to generate a first output;

(B″) measuring a characteristic of the first output;

and wherein steps (D) and (E) are:

(D) measuring the characteristic of the output relative to the characteristic of the first output; and
(E) relating the characteristic of the output relative to the characteristic of the first output to the extent of aggregation of the protein.

Steps (B′) and (B″) may be performed at start-up. The results may be stored electronically (e.g. as calibration data).

In a preferred embodiment, the sensor device is an interferometric sensor device. The sensor component of the interferometric sensor device may comprise at least one waveguide (e.g. a slab or channel waveguide) or a fibre optic component. For example, the sensor component may be a waveguide structure.

The waveguide structure may be generally of the planar type disclosed in WO-A-98/22807 or WO-A-01/36945. The response of the characteristic of the output of a sensor component of this type depends critically upon the extent of aggregation. Moreover by exploiting a sensor device of this type, relatively small samples of bodily fluid are required making it possible to advantageously utilise less invasive physiological fluid sampling (e.g. subcutaneous or transcutaneous sampling) or to be advantageously used in situations where large vascular blood samples are difficult to extract.

Preferably the sensor component is a waveguide structure including:

either (a) one or more sensing layers capable of inducing in a secondary waveguide a measurable response to a change in the localised environment caused by the introduction of the fluid;

or (b) a sensing waveguide capable of exhibiting a measurable response to a change in the localised environment caused by the introduction of the fluid.

In this embodiment, the introduction of the fluid contributes to a change in the effective refractive index of the sensor component. The waveguide structure is particularly sensitive to changes in molecular density and this is advantageously exploited to determine the extent of aggregation of the protein. By way of example, where an antibody is the specific binding partner, specific binding of a monomeric or lowly aggregated protein typically gives rise to a significant change in molecular density (e.g. an increase). On the other hand, specific binding of a highly aggregated protein typically gives rise to a less significant change in molecular density (e.g. a decrease).

Particularly preferably the sensor component is a waveguide structure including:

either (a) one or more sensing layers capable of inducing in a secondary waveguide a measurable response to a change in the localised environment caused by the introduction of the fluid and an inactive (e.g. deactivated) secondary waveguide in which the sensing layer is incapable of inducing a measurable response to a change in the localised environment caused by the introduction of the fluid or (b) a sensing waveguide capable of exhibiting a measurable response to a change in the localised environment caused by the introduction of the fluid and an inactive (e.g. deactivated) waveguide substantially incapable of exhibiting a measurable response to a change in the localised environment caused by the introduction of the fluid.

Preferably each of the sensing waveguide or secondary waveguide (or any additional waveguides such as reference waveguides) of the sensor component is a planar waveguide (ie a waveguide which permits light propagation in any arbitrary direction within the plane). Particularly preferably each planar waveguide is a slab waveguide.

Preferably the sensor component constitutes a multi-layered structure (e.g. a laminated waveguide structure) of the types disclosed in WO-A-98/22807, WO-A-01/36945 and WO-A-01/36946 (Farfield Sensors Limited). In a preferred embodiment, each of the plurality of layers in the multi-layered sensor component are built onto a substrate (e.g. of silicon) through known processes such as PECVD, LPCVD, etc. Intermediate transparent layers may be added (e.g silicon dioxide) if desired. Typically the sensor component is a multilayered structure of thickness in the range 0.2-10 microns. A layered structure advantageously permits layers to be in close proximity (e.g. a sensing waveguide and an inactive (reference) waveguide may be in close proximity to one another so as to minimise the deleterious effects of temperature and other environmental factors). Preferably the sensor component comprises a stack of transparent dielectric layers wherein layers are placed in close proximity. Preferably each layer is fabricated to allow equal amounts of electromagnetic radiation to propagate by simultaneous excitation of the guided modes in the structure.

The characteristic of the output may be a positional characteristic. Preferably the output is a pattern of interference fringes which may be measured (step (D)) by a conventional measuring means (see for example WO-A-98/22807) e.g. one or more detectors such as photodetectors which measure the intensity of electromagnetic radiation. Preferably step (D) comprises: measuring movements in the pattern of interference fringes. Particularly preferably step (D) further comprises: calculating the phase shift from the movements in the pattern of interference fringes.

Preferably the sensor component is adapted so as to be usable in evanescent mode or whole waveguide mode.

Thus in a first embodiment, the sensor component includes one or more sensing layers capable of inducing in a secondary waveguide a measurable response to a change in the localised environment caused by the introduction of the fluid. In this first embodiment, the sensor device is advantageously adapted to optimise the evanescent component so as to induce in the secondary waveguide a measurable response.

The desired antibody may be attached to or absorbed in the sensing layer which itself may be obtained by derivatising the surface of the sensor component. For example, the sensing layer may comprise an absorbent material (e.g. a polymeric material such as polymethylmethacrylate, polysiloxane, poly-4-vinylpyridine). For example, the sensing layer may comprise a porous silicon material (e.g. trimethoxyaminosilane) capable of being biofunctionalised with the specific binding partner.

In a preferred method of the invention, the secondary waveguide comprises silicon oxynitride or silicon nitride.

In a second embodiment, the sensor component includes a sensing waveguide capable of exhibiting a measurable response to a change in the localised environment caused by the introduction of the fluid. In this second embodiment, the sensor device is adapted to minimise the evanescent component and may be used advantageously in whole waveguide mode.

The desired antibody may be attached to or absorbed in the sensing waveguide which itself may be obtained by derivatising the surface of the sensor component. For example, the sensing waveguide may comprise an absorbent material (e.g. a polymeric material such as polymethylmethacrylate, polysiloxane, poly-4-vinylpyridine). For example, the sensing waveguide may comprise a porous silicon material (e.g. trimethoxyaminosilane) capable of being biofunctionalised with the specific binding partner.

To optimise the performance of the first embodiment, the sensor component may further comprise an inactive secondary waveguide in which the sensing layer is incapable of inducing a measurable response to a change in the localised environment caused by the introduction of the fluid. The inactive secondary waveguide is capable of acting as a reference layer. It is preferred that the secondary waveguide and inactive secondary waveguide have identical properties with the exception of the response to the change in the localised environment caused by the introduction of the fluid. By way of example, the secondary waveguide and inactive secondary waveguide are made of silicon oxynitride.

To optimise the performance of the second embodiment, the sensor component may further comprise an inactive (e.g. deactivated) waveguide substantially incapable of exhibiting a measurable response to a change in the localised environment caused by the introduction of the fluid. The inactive waveguide is capable of acting as a reference layer. The physical, biological and chemical properties of the sensing waveguide and inactive waveguide are as similar as possible (with the exception of the response to the change in the localised environment caused by the introduction of the fluid). Typically the inactive waveguide is made of silicon oxynitride.

As a consequence of the introduction of the fluid, changes in the dielectric properties (e.g. the effective refractive index) of the sensing waveguide or sensing layer occur. This causes a measurable response (ie a change in the transmission of electromagnetic radiation down the sensing waveguide (or waveguides) in whole waveguide mode or the secondary waveguide in evanescent field mode) which (in one embodiment) manifests itself as a movement of interference fringes. This differs according to whether the sensor component is interrogated in transverse electric (TE) or transverse magnetic (TM) mode.

By way of example, the movement of the pattern of interference fringes may be used to calculate the phase shift which takes place in the sensing waveguide or sensing layer during the passage of electromagnetic radiation through the sensor component. The phase shift is effectively directly proportional to changes occurring in the effective refractive index of the sensing waveguide or sensing layer and differs according to whether the sensor component is interrogated in TE or TM mode.

A pattern of interference fringes (e.g. for TE and TM modes respectively) may be generated when the electromagnetic radiation from the sensor component is coupled into free space and may be recorded in a conventional manner (see for example WO-A-98/22807). A response of the sensor component to a change in the localised environment may be measured from movement of the fringes in the interference pattern. The phase shift of the electromagnetic radiation in the sensor component (e.g. induced in the secondary waveguide in evanescent field mode or exhibited in the sensing waveguide in whole waveguide mode) may be calculated. In turn, the change in molecular density contributing to a change in the effective refractive index may be calculated.

Movement in the interference fringes may be measured either using a single detector which measures changes in the intensity of electromagnetic radiation or a plurality of such detectors which monitor the change occurring in a number of fringes or the entire interference pattern. The one or more detectors may comprise one or more photodetectors (e.g. photodiodes). Where more than one photodetector is used this may be arranged in an array. In an array format, the relating means capable of relating the measurable response in TM mode and the measurable response in TE mode to the introduction of the fluid may be deployed in a spatially resolved manner. Such spatial resolution can be achieved by means of (for example) remote imaging or lithography or scanning a measurement probe as in the case of an atomic microprobe.

In a preferred embodiment of the method of the invention, step (B) is carried out with electromagnetic radiation in TM mode.

In a preferred embodiment of the method of the invention, step (B) is carried out with electromagnetic radiation in TE mode.

In a preferred embodiment of the method of the invention, step (B) comprises:

(B1) irradiating the sensor component with electromagnetic radiation in TE mode to produce a first pattern of interference fringes;

(B2) irradiating the sensor component with electromagnetic radiation in TM mode to produce a second pattern of interference fringes;

and step (D) comprises:

(D1) measuring movements in the first pattern of interference fringes; and

(D2) measuring movements in the second pattern of interference fringes.

Particularly preferably step (D) of the method of the invention further comprises:

(D3) calculating the phase shift of the sensor component in TM mode from the movements in the first pattern of interference fringes;

(D4) calculating the phase shift of the sensor component in TE mode from the movements in the second pattern of interference fringes;

and step (E) is

relating the phase shift of the sensor component in TM mode and the phase shift of the sensor component in TE mode to the extent of aggregation of the protein.

More preferably step (D) of the method of the invention further comprises:

(D3) calculating the phase shift of the sensor component in TM mode from the movements in the first pattern of interference fringes

(D4) calculating the phase shift of the sensor component in TE mode from the movements in the second pattern of interference fringes;

(D5) calculating the phase shift of the sensor component in TM mode relative to the phase shift of the sensor component in TE mode;

and step (E) is

relating the phase shift of the sensor component in TM mode relative to the phase shift of the sensor component in TE mode to the extent of aggregation of the protein.

Preferably the phase shift of the sensor component in TM mode relative to the phase shift of the sensor component in TE mode is a ratio of the phase shift of the sensor component in TM mode to the phase shift of the sensor component in TE mode.

The extent of aggregation of the protein is typically determined qualitatively but may be determined quantitatively in terms of a change in molecular density or mass.

Thus in a preferred embodiment, the method further comprises:

(F1) relating the response of the characteristic of the output to a change in the intrinsic refractive index and/or the volume; and

(F2) calculating the change in the molecular density; and

(F3) optionally calculating the change in mass.

Preferably the method further comprises:

(F1) relating the movements in the first pattern of interference fringes and second pattern of interference fringes to a change in the intrinsic refractive index and/or the volume; and

(F2) calculating the change in the molecular density; and

(F3) optionally calculating the change in mass.

In a particularly preferred embodiment, step (F1) comprises:

(F1) relating the movements in the first pattern of interference fringes and second pattern of interference fringes to a change in the intrinsic refractive index and/or the thickness of the sensing layer or sensing waveguide.

Preferably the sensor device further comprises:
relating means capable of relating the measurable response to the extent of aggregation of the protein.

The relating means enables the measurable response to be directly related to the extent of aggregation of the protein or indirectly related to the extent of aggregation of the protein (e.g. to the dimensionality such as the volume and to the mass) and may be for example a piezoelectric sensing system (where the measured resonant frequency is related to the mass and the decay time constant of free oscillation to the dimension) or an optical system such as a surface plasmon resonance device in combination with a device based on other analytical techniques such as ellipsometry.

Where volume is to be measured directly or indirectly, this may be by determination of one or more of the relevant dimensions or by displacement of the medium surrounding the volume.

In a preferred embodiment of the method, said sensor device further comprises:

first irradiating means for irradiating the sensor component with electromagnetic radiation in TM mode;

second irradiating means for irradiating the sensor component with electromagnetic radiation in TE mode;

measuring means for measuring the measurable response of the sensor component in TM mode and for measuring the measurable response of the sensor component in TE mode; and

relating means capable of relating the measurable response of the sensor component in TM mode and the measurable response of the sensor component in TE mode to the extent of aggregation of the protein.

The first and second irradiating means may be the same or different. The measuring means may be one or more detectors in an array.

Preferably the sensor device further comprises:

a synchronising means for synchronising the measuring means with the first irradiating and second irradiating means so as to correlate the measurement of the measurable response of the sensor component in TE mode and of the measurable response of the sensor component in TM mode with the irradiation of the sensor component with electromagnetic radiation in TE and TM mode respectively.

Particularly preferably the synchronising means is capable of calculating the phase shift of the sensor component in TE mode and the phase shift of the sensor component in TM mode.

Particularly preferably the synchronising means is capable of relating the movements in the first pattern of interference fringes and second pattern of interference fringes to the extent of aggregation of the protein.

The first and second irradiating means may be adapted to irradiate the sensor component with electromagnetic radiation in TE or TM mode sequentially or simultaneously. The first and second irradiating means may be the same or different. Where different sources of electromagnetic radiation are used, an optical switch (e.g. a rotating mirror) may be used to switch rapidly between the two. Alternatively, a single source of electromagnetic radiation may be used to simultaneously excite TE and TM modes of the sensor component by (for example) aligning the polarisation vector of the linearly polarised source at an angle with respect to the plane of the sensing waveguide or sensing layer of the sensor component. An active analyser system may be used to alternately remove the unwanted TE or TM mode radiation during data capture of the desired TM or TE output respectively. The active analyser system may comprise an electro-optic half wave plate placed in series with an analyser.

In a preferred embodiment, an adjustable analyser may be used to measure the first pattern of interference fringes and the second pattern of interference fringes separately. The measurements may be synchronised with the excitation and/or polarisation procedure to ensure that phase shift information is correlated with TE and TM excitation.

A controller may be provided to synchronise the one or more sources of electromagnetic radiation and one or more detectors. For example, the controller may capture the data from a photodetector (e.g. photodiode) array. The firing of the (or each) source of electromagnetic radiation may be synchronised by the controller with the alternate capture of the first and second pattern of interference fringes generated in TM mode and TE mode. The controller may be adapted to calculate the phase shift in TE and TM modes independently.

Electromagnetic radiation generated from a conventional source may be propagated into the sensor component in a number of ways. In a preferred embodiment, electromagnetic radiation is simply input via an end face of the sensor component (this is sometimes described as “an end firing procedure”). Preferably the electromagnetic radiation source provides incident electromagnetic radiation having a wavelength falling within the optical range. Propagating means may be employed for substantially simultaneously propagating incident electromagnetic radiation into a plurality of waveguides. For example, one or more coupling gratings or mirrors may be used. A tapered end coupler rather than a coupling grating or mirror may be used to propagate radiation into the lowermost waveguide. Preferably the amount of electromagnetic radiation in the sensing waveguide/inactive waveguide or in the secondary waveguide/inactive secondary waveguide is equal.

The incident electromagnetic radiation may be oriented (e.g. plane polarised) as desired using an appropriate polarising means. The incident electromagnetic radiation may be focussed if desired using a lens or similar micro-focussing means.

A plurality of electromagnetic radiation detector units (e.g. in an array) and/or a plurality of electromagnetic radiation sources may be used to measure in discrete areas of the sensor component simultaneously the responses to changes in the localised environment caused by the introduction of the fluid. Alternatively, the position of the electromagnetic radiation detector and electromagnetic radiation source relative to the sensor component may be changed to provide information concerning responses in discrete areas of the sensor component.

Preferably the sensor device comprises:

means for intimately exposing at least a part of the (or each) sensing layer or the sensing waveguide of the sensor component to the localised environment.

In a preferred embodiment, the means for intimately exposing at least a part of the sensing layer or the sensing waveguide to the localised environment is integrated onto the sensor component.

Preferably the means for intimately exposing at least a part of the (or each) sensing layer or the sensing waveguide of the sensor component to the localised environment is as described in WO-A-01/36945. The means may be automated in order to reduce the requisite degree of user intervention.

The means for intimately exposing at least a part of the (or each) sensing layer or the sensing waveguide to the localised environment may be a part of a microstructure positionable on the surface of and in intimate contact with the sensor component.

Preferably the microstructure comprises means for intimately exposing at least a part of the sensing layer or the sensing waveguide to the localised environment in the form of one or more microchannels and/or microchambers. For example, the fluid may be fed through microchannels or into the microchannels by capillary action or positively fed by an urging means.

In a preferred embodiment, the means for intimately exposing at least a part of the (or each) sensing layer or the sensing waveguide to the localised environment is included in a cladding layer. For example, microchannels and/or microchambers may be etched into the cladding layer. The cladding layer may perform optical functions such as preventing significant discontinuities at the boundary of the sensing waveguide or sensing layer(s) or chemical functions such as restricting access of certain species to the sensing waveguide or sensing layer(s). The cladding layer may be integrated onto the sensor component.

Preferably the whole of or a portion of any additional functionality may be included in the cladding layer. In one embodiment, the sensing layer itself may be incorporated in the cladding layer (for example in the form of an absorbent material). Particularly preferably, the whole of the additional functionality may be provided in the cladding layer and include sensing devices such as for example quadrature electric field tracks or other microfluidic sensing devices. The cladding layer may incorporate an electromagnetic source (e.g. a laser) and/or means for detecting electromagnetic radiation (of the type detailed below). The cladding layer may incorporate a chemical separating means (e.g. an HPLC based device).

Preferably the means for exposing at least a part of the (or each) sensing layer or the sensing waveguide of the sensor component to the localised environment is a sensor cell. Preferably the sensor cell has a low volume (e.g. 50 microlitres or less).

Viewed from a yet further aspect the present invention provides a kit of parts comprising:

a sensor device for determining the extent of aggregation of a protein in a fluid having a sensor component capable of exhibiting a measurable response to a change in a localised environment caused by the introduction of the fluid; and

a specific binding partner capable of binding to a surface of said sensor component so as to render the measurable response to a change in a localised environment discriminatory to the extent of aggregation of the protein.

The present invention will now be described in a non-limitative sense with reference to the Examples and accompanying Figures in which:

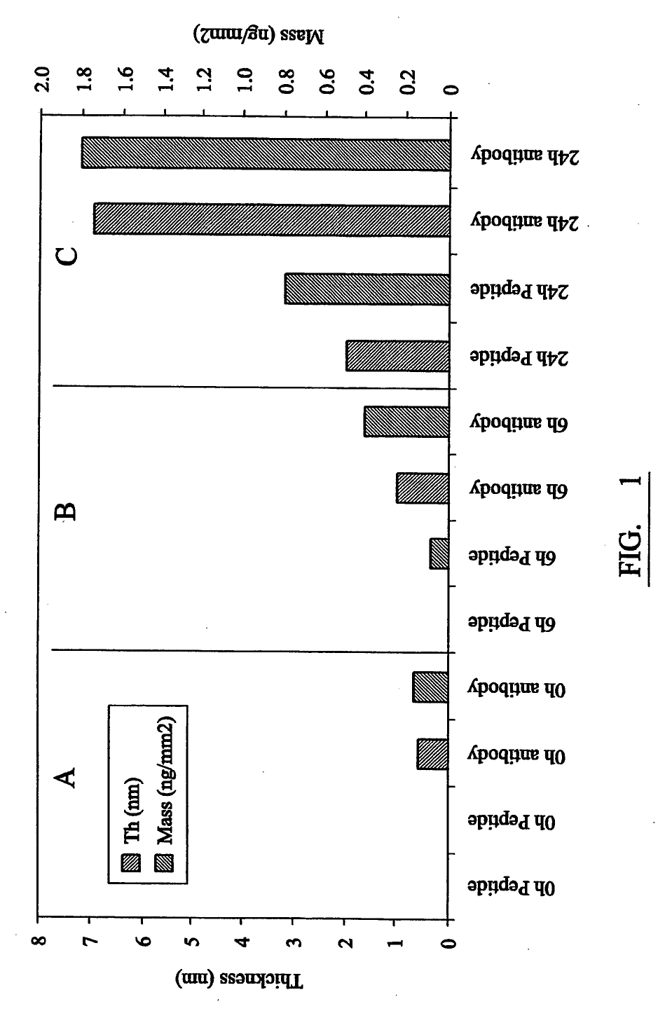

FIG. 1 illustrates thickness and mass changes in a biological sensing layer containing 6E10, binding fresh protein and aggregated protein in direct and sandwich assay formats; and

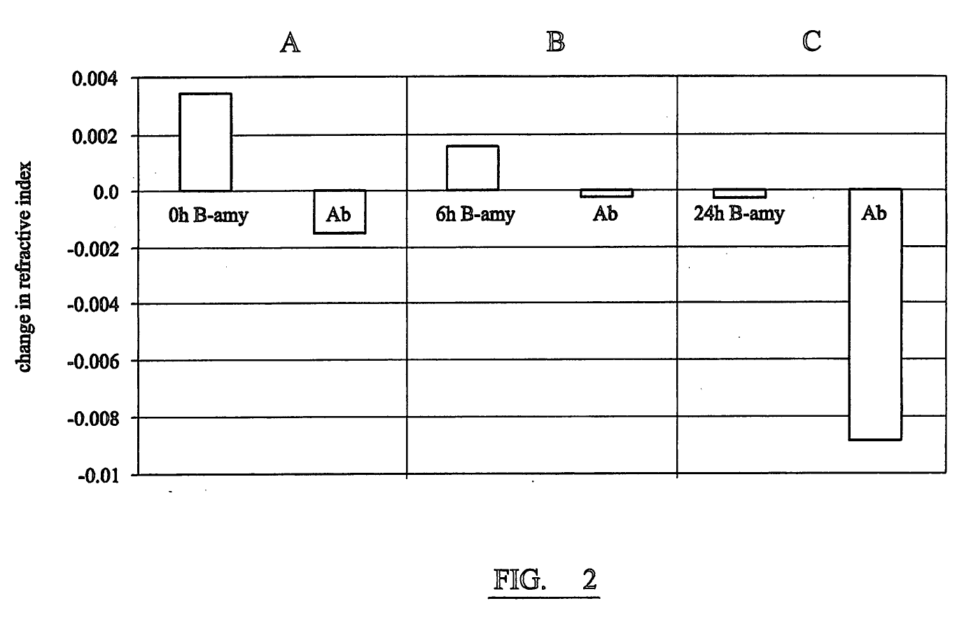

FIG. 2 illustrates density changes in a biological sensing layer containing 6E10, binding fresh protein and aggregated protein in direct and sandwich assay formats.

Sensor Device Preparation

The surface of a silicon oxynitride sensor device (of the type described in WO98/22807) was derivatised to obtain a sensing layer using trimethoxyaminosilane, perfused with a 2 mg/ml PBS solvated solution of EZ-LINK™ n-hydroxysuccinimido-LC-biotin for 5 minutes and then incubated for 10 minutes to produce a biotinylated sensing layer. The LC section gives a spacer arm length of 224 nm so as to allow increased binding of protein. Streptavidin (40 μg/ml in PBS) was added to provide a surface with free biotin binding sites available for further binding of functional biomolecules. The commercially available antibody biotinylated 6E-10 (Ab) dissolved in PBS at a concentration of 80 μg/ml was flowed over the surface for 5 minutes and incubated for 10 minutes. The sensor surface was then blocked with a solution of TWEEN®, gelatine and gamma globulin. The blocking solution was incubated on the surface then eluted to a stable baseline. The sensor device was then ready to be used for testing.

Testing

In a first (direct assay) test, the sensor device was subjected to a 50 μM solution of β-1-40 amyloid (AB) which was obtained from synthetic sources (A) as a fresh preparation, (B) as a solution which had been allowed to aggregate for 6 hours and (C) as a solution which had been allowed to aggregate for 24 hours (having been determined by cell culture to be close to maximum toxicity). For each of the three preparations, the thickness and mass changes are denoted in FIG. 1 by “xh peptide” (where x=0, 6 or 24) and the density changes in FIG. 2 by “xh B-amy”.

In a second (sandwich assay) test, the sensor device was further subjected to an additional amount of Ab. For each of the three preparations, the thickness and mass changes are denoted in FIG. 1 by “xh antibody” (where x=0, 6 or 24) and the density changes in FIG. 2 by “Ab”

The sensor device was interrogated with TE and TM mode irradiation and for the first and second test, the data was analysed as described in WO-A-01/36946 and WO-A-095365.

In the case of the first test of the fresh preparation (A), the thickness of the sensing layer increased very slightly but the density increased quite significantly as the antibody bound the AB which is either monomeric or has aggregated to a very small extent. In the case of the 24 hour aggregated solution (C), the sensing layer increased in thickness significantly in the first and second test but the density decreased (very significantly with the second test). This was consistent with the antibody binding AB which had aggregated to form large molecules which (relative to the fresh preparation) are incapable of packing on the sensor surface efficiently.

1. A sensor device for determining the extent of aggregation of a protein in a fluid having a sensor component capable of exhibiting a measurable response to a change in a localised environment caused by the introduction of the fluid, wherein a surface of said sensor component exposed to the fluid is provided with a specific binding partner to the protein rendering the measurable response to the change in the localised environment discriminatory to the extent of aggregation of the protein.

2. A sensor device as claimed in claim 1 wherein the protein is beta amyloid.

3. A sensor device as claimed in claim 2 wherein the specific binding partner to beta amyloid is an optionally biotinlyated antigen.

4. A sensor device as claimed in any preceding claim wherein the fluid is a bodily fluid.

5. A sensor device as claimed in any preceding claim wherein the sensor component is capable of exhibiting a measurable response in a parameter selected from effective refractive index, a dielectric constant, a viscoelastic property, a frequency of oscillation, a thermal absorption/desorption parameter, the permeability, the absorption of energy or of energetic particles or the particle size.

6. A sensor device as claimed in claim 5 wherein the parameter is the effective refractive index.

7. A sensor device as claimed in any preceding claim wherein the sensor component is derivatised for the purposes of attaching or absorbing the specific binding partner.

8. A sensor device as claimed in claim 7 wherein the surface of the sensor component comprises an absorbent material.

9. A sensor device as claimed in claim 7 wherein the surface of the sensor component comprises a porous silicon material capable of being biofunctionalised with the specific binding partner.

10. A sensor device as claimed in any preceding claim wherein the sensor component is a waveguide structure including:
either (a) one or more sensing layers capable of inducing in a secondary waveguide measurable response to a change in the localised environment caused by the introduction of the fluid;
or (b) a sensing waveguide capable of exhibiting a measurable response to a change in the localised environment caused by the introduction of the fluid.

11. A sensor device as claimed in any preceding claim wherein the sensor component is a waveguide structure including:
either (a) one or more sensing layers capable of inducing in a secondary waveguide a measurable response to a change in the localised environment caused by the introduction of the fluid and an inactive secondary waveguide in which the sensing layer is incapable of inducing a measurable response to a change in the localised environment caused by the introduction of the fluid or (b) a sensing waveguide capable of exhibiting a measurable response to a change in the localised environment caused by the introduction of the fluid and an inactive waveguide substantially incapable of exhibiting a measurable response to a change in the localised environment caused by the introduction of the fluid.

12. A sensor device as claimed in claim 10 or 11 wherein the sensing waveguide or secondary waveguide of the sensor component is a planar waveguide.

13. A sensor device as claimed in claim 12 wherein each planar waveguide is a slab waveguide.

14. A method for determining the extent of aggregation of a protein in a fluid, said method comprising:
(A) providing a sensor device as defined in any preceding claim;
(B) irradiating the sensor component with electromagnetic radiation to generate an output;
(C) introducing the fluid into the localised environment;
(D) measuring the response of a characteristic of the output; and
(E) relating the response of the characteristic of the output to the extent of aggregation of the protein.

15. A method as claimed in claim 14 wherein step (C) further comprises:
(C′) introducing an amount of the specific binding partner into the localised environment.

16. A method as claimed in claim 14 or 15 wherein step (B) comprises:
(B′) irradiating the sensor component with electromagnetic radiation to generate a first output;
(B″) measuring a characteristic of the first output;
and wherein steps (D) and (E) are:
(D) measuring the characteristic of the output relative to the characteristic of the first output; and
(E) relating the characteristic of the output relative to the characteristic of the first output to the extent of aggregation of the protein.

17. A method as claimed in any of claims 14 to 16 wherein the characteristic of the output is a positional characteristic.

18. A method as claimed in any of claims 14 to 17 wherein the output is a pattern of interference fringes.

19. A method as claimed in claim 18 wherein step (D) comprises: measuring movements in the pattern of interference fringes.

20. A method as claimed in claim 19 wherein step (D) further comprises: calculating the phase shift from the movements in the pattern of interference fringes.

21. A method as claimed in any of claims 18 to 20 wherein step (B) comprises:
(B1) irradiating the sensor component with electromagnetic radiation in TE mode to produce a first pattern of interference fringes;
(B2) irradiating the sensor component with electromagnetic radiation in TM mode to produce a second pattern of interference fringes; and step (D) comprises:
(D1) measuring movements in the first pattern of interference fringes; and
(D2) measuring movements in the second pattern of interference fringes.

22. A method as claimed in claim 21 wherein step (D) further comprises:
(D3) calculating the phase shift of the sensor component in TM mode from the movements in the first pattern of interference fringes;
(D4) calculating the phase shift of the sensor component in TE mode from the movements in the second pattern of interference fringes;
and step (E) is
relating the phase shift of the sensor component in TM mode and the phase shift of the sensor component in TE mode to the extent of aggregation of the protein.

23. A method as claimed in claim 22 wherein step (D) further comprises:
(D3) calculating the phase shift of the sensor component in TM mode from the movements in the first pattern of interference fringes
(D4) calculating the phase shift of the sensor component in TE mode from the movements in the second pattern of interference fringes;
(D5) calculating the phase shift of the sensor component in TM mode relative to the phase shift of the sensor component in TE mode;
and step (E) is
relating the phase shift of the sensor component in TM mode relative to the phase shift of the sensor component in TE mode to the extent of aggregation of the protein.

24. A method as claimed in claim 23 wherein the phase shift of the sensor component in TM mode relative to the phase shift of the sensor component in TE mode is a ratio of the phase shift of the sensor component in TM mode to the phase shift of the sensor component in TE mode.

25. A method as claimed in any of claims 14 to 24 further comprising:
(F1) relating the response of the characteristic of the output to a change in the intrinsic refractive index and/or the volume;
(F2) calculating the change in the molecular density; and
(F3) optionally calculating the change in mass.

26. A kit of parts comprising:
a sensor device for determining the extent of aggregation of a protein in a fluid having a sensor component capable of exhibiting a measurable response to a change in a localised environment caused by the introduction of the fluid; and
a specific binding partner capable of binding to a surface of said sensor component so as to render the measurable response to a change in a localised environment discriminatory to the extent of aggregation of the protein.
